(19) United States
(12) Patent Application Publication
(10) Pub. No.: US 2004/0198652 A1
(43) Pub. Date: Oct. 7, 2004

(54) Methods and compositions for preventing and treating septic shock and endotoxemia

(76) Inventor: J. Paul Carter, North Andover, MA (US)

Correspondence Address: CLARK & ELBING LLP, 101 FEDERAL STREET, BOSTON, MA, 02110, US

(21) Appl. No.: 10/476,318

(22) PCT Filed: Apr. 19, 2002
(86) PCT No.: PCT/US02/12504

Related U.S. Application Data
(60) Provisional application No. 60/286,215, filed on Apr. 24, 2001.

(51) Int. Cl. A61K 38/17; A61K 31/739

(52) U.S. Cl. 514/12; 514/54

(57) ABSTRACT

The invention provides methods and compositions for treating septic shock, endotoxemia, and related diseases and conditions, involving the use of an antiendotoxin drug and an activated Protein C.

### BACKGROUND OF THE INVENTION

This invention relates to methods and compositions for use in preventing and treating septic shock and endotoxemia.

Since the 1930's, the increasing use of immunosuppressive therapy and invasive devices, as well as the increased incidence of antibiotic resistance in bacteria, have led to a gradual rise in the occurrence of sepsis and septic shock. Currently, the estimated incidences in the U.S. of sepsis and septic shock are 400,000 and 200,000 patients/year, respectively. This results in about 100,000 fatalities/year, making septic shock the most common non-coronary cause of death in the hospital Intensive Care Unit (ICU). Currently, ICU therapy for septic shock is limited to antibiotic therapy, cardiovascular resuscitation, vasopressor/ionotrope therapy, and ventilatory support. This ICU care can cost up to $1,500/day and average a total of $13,000 to $30,000 per patient. Clearly, any therapy that can reduce the morbidity and therefore the cost of care in sepsis/septic shock is of great value.

It is likely that antibiotics themselves can worsen morbidity associated with sepsis. Their bactericidal action can result in the release of endotoxin from gram-negative bacteria, which are believed to induce many pathophysiological events, such as fever, shock, disseminated intravascular coagulation (DIC), and hypotension. Consequently, medicines for the treatment of gram-negative sepsis have been desired for some time, especially drugs capable of blocking endotoxin or cytokines derived from endotoxin-mediated cellular stimulation. To this end, various strategies for treatment have included administration of antibodies or other agents against lipopolysaccharide (LPS) or cytokines, such as INF-α and interleukin-1. For various reasons, these approaches have failed.

While endotoxin itself is a highly heterogenous molecule, the expression of many of the toxic properties of endotoxin is attributed to a highly conserved hydrophobic lipid A portion. An effective drug that acts as an antagonist to this conserved structure is known as E5564 (also known as compound 1287 and SGEA). This drug is described as compound 1 in U.S. Pat. No. 5,935,938, which is hereby incorporated by reference.

Activated protein C (aPC) is a serine protease that plays a central role in down-regulating blood coagulation, resulting in protection against thrombosis, and also has anti-inflammatory and fibrinolytic activities. Human protein C is produced primarily in the liver as a single, inactive polypeptide of 461 amino acids. This precursor molecule undergoes multiple post-translational modifications to yield a two-chain circulating zymogen, which is activated in vivo by thrombin cleavage.

### SUMMARY OF THE INVENTION

The invention provides methods of preventing or treating septic shock, endotoxemia, and related diseases or conditions (e.g., systemic inflammatory response syndrome (SIRS), sepsis, or septicemia) in a patient (e.g., a human patient) by administering an antiendotoxin compound and an activated Protein C (e.g., a recombinant human aPC) to the patient. The drugs can be administered to the patient using any standard method including, for example, continuous infusion, by bolus, or intermittent infusion. The antiendotoxin compound can be a Lipid A analog. For example, the antiendotoxin compound can be of the formula:

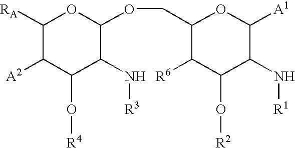

where R¹ is selected from the group consisting of

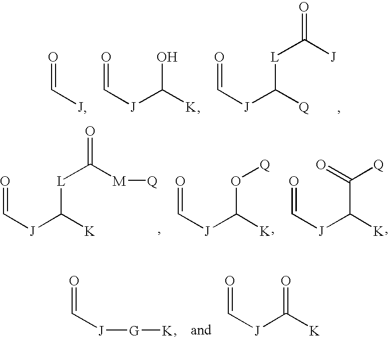

where each of J, K, and Q, independently, is straight or branched C1 to C15 alkyl;

L is O, NH, or CH₂; M is O or NH; and G is NH, O, S, SO, or SO₂;

R² is straight or branched C5 to C15 alkyl;

R³ is selected from the group consisting of straight or branched C5 to C18 alkyl,

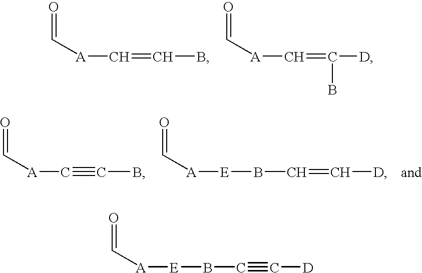

where E is NH, O, S, SO, or SO₂; each of A, B, and D, independently, is straight or branched C1 to C15 alkyl;

R⁴ is selected from the group consisting of straight or branched C4 to C20 alkyl, and

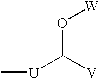

where each of U and V, independently, is straight or branched C2 to C15 alkyl and W is hydrogen or straight or branched C1 to C5 alkyl;

R_{A} is R⁵ or R⁵—O—CH₂—, R⁵ being selected from the group consisting of hydrogen, J′, -J′-OH, -J′-O-K′, -J′-O-K′-OH, and -J′-O—PO(OH)₂, where each of J′ and K′, independently, is straight or branched C1 to C5 alkyl;

R⁶ is selected from the group consisting of hydroxy, halogen, C1 to C5 alkoxy, and C1 to C5 acyloxy;

A¹ and A², independently, are selected from the group consisting of OH,

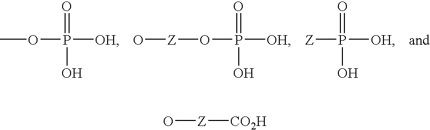

where Z is straight or branched C1 to C10 alkyl;

or a pharmaceutically acceptable salt thereof.

As a specific example, the antiendotoxin compound can have the following structure:

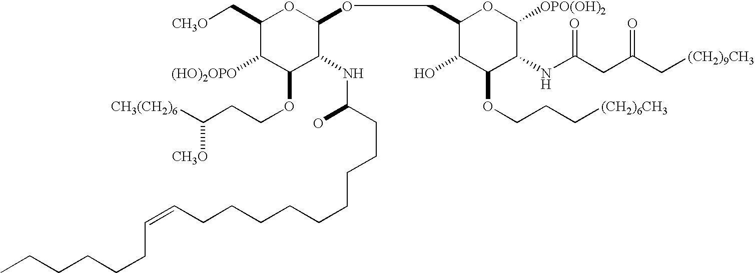

The invention also includes compositions including the compounds described above, as well as the use of these compounds in the preparation of medicaments for preventing or treating diseases or conditions related to endotoxemia, as discussed herein.

Patients that can be treated using the methods and compositions of the invention include, for example, surgical patients (e.g., cardiac surgical patients), if appropriate, patients that have or are at risk of developing endotoxemia, sepsis, or septic shock, patients that are infected with HIV, and patients who are immunocompromised due to their suffering from an immunological disorder.

The invention also includes use of the above-described agents in the prevention or treatment of septic shock or endotoxemia (see above), as well as use of these agents in the preparation of medicaments for these purposes.

The methods of the invention provide significant therapeutic benefits, and are easily carried out, especially with many of the patients treated according to the methods of the invention, who already have intravenous lines inserted, as part of their treatment in the ICU. Also, aPC and antiendotoxin drugs have different and complementary modes of action, thus enabling their combined use to result in synergistic effects. Other features and advantages of the invention will be apparent from the following detailed description and the claims.

### DETAILED DESCRIPTION

The invention provides methods of preventing or treating septic shock, endotoxemia, and related diseases or conditions by administration of an antiendotoxin drug (e.g., a Lipid A analog; see below) and an activated Protein C (aPC). The invention also provides pharmaceutical compositions including an antiendotoxin drug and an aPC that can be used in such methods. The methods and compositions of the invention are described in further detail, as follows.

Antiendotoxin compounds that can be used in the methods and compositions of the invention include, for example, Lipid A analogs, such as Compound 1287 (SGEA; EE564; U.S. Pat. No. 5,935,938; see structure, above) and Compound B531 (U.S. Pat. No. 5,530,113), as well as other compounds described in these patents and the following U.S. patents: U.S. Pat. No. 5,612,476, U.S. Pat. No. 5,756,718, U.S. Pat. No. 5,843,918, U.S. Pat. No. 5,750,664, and U.S. Pat. No. 5,681,824.

Activated Protein C for use in the invention can be obtained from any of a number of sources, using any of several standard methods. For example, the aPC can be purified from plasma (e.g., human plasma). Preferably, however, the aPC used in the invention is produced using recombinant methods. For example, the aPC (e.g., human aPC) can be produced using eukaryotic cell culture systems (e.g., human kidney 293, HEPG-2, LLC-MK2, CHO, or AV12 cells), transgenic animals, transgenic plants, or in vitro systems. In these systems, the protein can be produced as an inactive precursor, which, after purification, is activated by thrombin cleavage and formulated for administration. Alternatively, the aPC can be produced by direct secretion of the activated form of Protein C.

Details of producing, purifying, activating, and formulating aPC are known in the art and are described, for example, in U.S. Pat. No. 6,156,734, which is incorporated by reference herein in its entirety. Also, aPC genes and plasmids that can be used in these methods are described in U.S. Pat. Nos. 4,981,952; 4,775,624; and 4,992,373, which are also incorporated by reference herein. Activated Protein C can also be obtained from commercial sources. For instance, a specific example of an aPC that can be used in the invention is produced by Eli Lilly and Company, under the name of ZOVANT™ (activated Protein C). Derivatives of aPC, such as those described in U.S. Pat. Nos. 5,453,373 and 5,516,650, which are incorporated by reference herein, can also be used in the invention.

Administration of the antiendotoxin drug and aPC can be carried out using any of several standard methods including, for example, continuous infusion, bolus injection, intermittent infusion, or combinations of these methods. The drugs can be administered together in a single solution (e.g., a composition as described herein) or separately and, when administered separately, the time periods of administration of the drugs can overlap, partially overlap, or not overlap, as determined to be appropriate by one of skill in this art.

A mode of administration of both drugs is by continuous intravenous infusion. In such an approach, the infusion dosage rate of the antiendotoxin drug can be, for example, 0.001-0.5 mg/kg body weight/hour, more preferably 0.01-0.2 mg/kg/hour, and most preferably 0.03-0.1 mg/kg/hour, infused over the course of, for example, 12-100, 60-80, or about 96 hours. The infusion of antiendotoxin drug can, if desired, be preceded by a bolus injection; preferably, such a bolus injection is given at a dosage of 0.001-0.5 mg/kg. Preferably, the total amount of antiendotoxin drug administered to a patient is 25-600 mg of drug, more preferably 35-125 mg, by infusion over a period of 60-100 hours.

Activated Protein C can be administered by continuous infusion at a dosage rate of, for example, 20-50 μg/kg/hour, more preferably 22-40 μg/kg/hour, and most preferably 24-30 μg/kg/hour, infused over the course of, for example, 24 to 144 hours. Similar to the antiendotoxin drug, continuous infusion of the aPC can be preceded, if desired by bolus administration of the aPC. For example, a portion (e.g., or ½) of the appropriate dose can be administered as a bolus injection for a time period of, e.g., 5 to 120 minutes, followed by continuous infusion for about 23 to 144 hours, resulting in proper dosage for that time period. Preferably, administration carried out so as to achieve a plasma level of about 2-200 ng/ml. As activity in the hospital, and particularly the ICU, is often hectic, minor variations in the time period of infusion of the drugs may occur and are also included in the invention.

Additional modes of administration of antiendotoxin drugs and an aPC, according to the methods of the invention, include bolus or intermittent infusion. For example, the drugs (either formulated together or separately) can be administered in a single bolus by intravenous infusion through, for example, a central access line or a peripheral venous line, or by direct injection, using a syringe. Such administration may be desirable if a patient is only at short-term risk for exposure to endotoxin, and thus does not need prolonged persistence of the drug. For example, this mode of administration may be desirable in surgical patients, if appropriate, such as patients having cardiac surgery, e.g., coronary artery bypass graft surgery and/or valve replacement surgery. In these patients, a single bolus infusion of, e.g., 0.10-15 mg/hour (e.g., 1-7 mg/hour or 3 mg/hour) of antiendotoxin drug can be administered over a period of four hours prior to and/or during surgery. (Note that the amount of drug administered is based on an assumed average weight of a patient of 70 kg.) Shorter or longer time periods of administration can be used, as determined to be appropriate by one of skill in this art, provided that the absolute amount of drug administered, as indicated above, is maintained.

In cases in which longer-term persistence of active drug is desirable, for example, in the treatment of a condition associated with long-term exposure to endotoxin, such as during infection or sepsis, or in appropriate surgical situations in which it is determined that prolonged treatment is desirable, intermittent administration can be carried out. In these methods, a loading dose is administered, followed by either (i) a second loading dose and a maintenance dose (or doses), or (ii) a maintenance dose or doses, without a second loading dose, as determined to be appropriate by one of skill in this art.

The first (or only) loading dose can be administered in a manner similar to that described for the single bolus infusion described above. That is, for antiendotoxin drug administration, 0.10-15 mg/hour (e.g., 3-7 mg/hour or 3 mg/hour) of drug can be administered to a patient over a period of four hours prior to surgery. (As is noted above, and is applicable throughout this description, the time periods of administration can be varied, provided that dosage levels are maintained.) If a second loading dosage is to be used, it can be administered about 12 hours after the initial loading dose, and can involve infusion of, e.g., 0.10-15 mg/hour (e.g., 1-7 mg/hour or 3 mg/hour) of drug over a period of, e.g., about two hours.

To achieve further persistence of active drug, a maintenance dose (or doses) of drug can be administered, so that levels of active drug are maintained in the blood of a patient. Maintenance doses can be administered at levels that are less than the loading dose(s), for example, at a level that is about ⅙ of the loading dose. Specific amounts to be administered in maintenance doses can be determined by a medical professional, with the goal that drug level is at least maintained. Maintenance doses can be administered, for example, for about 2 hours every 12 hours beginning at hour 24 and continuing at, for example, hours 36, 48, 60, 72, 84, 96, 108, and 120. Of course, maintenance doses can be stopped at any point during this time frame, as determined to be appropriate by a medical professional.

The methods and compositions of the invention can be used to prevent or to treat any of a large number of diseases and conditions associated with septic shock or endotoxemia. For example, the methods and compositions of the invention can be used in conjunction with any type of surgery or medical procedure, when appropriate, that could lead to the occurrence of endotoxemia or related complications (e.g., sepsis syndrome). As a specific example, the methods of the invention can be used in conjunction with cardiac surgery (e.g., coronary artery bypass graft, cardiopulmonary bypass, and/or valve replacement), transplantation (of, e.g., liver, heart, kidney, or bone marrow), cancer surgery (e.g., removal of a tumor), or any abdominal surgery. Additional examples of surgical procedures with which the methods of the invention can be used, when appropriate, are surgery for treating acute pancreatitis, inflammatory bowel disease, placement of a transjugular intrahepatic portosystemic stent shunt, hepatic resection, burn wound revision, and burn wound escharectomy. The methods of the invention can also be used in conjunction with non-surgical procedures in which the gastrointestinal tract is compromised. For example, the methods of the invention can be used in association with chemotherapy or radiation therapy in the treatment of cancer. The methods can also be used in the treatment of conditions associated with HIV infection, trauma, or respiratory distress syndrome, as well as with immunological disorders, such as graft-versus-host disease or allograft rejection.

As is noted above, the invention also includes compositions that include an antiendotoxin compound (e.g., a Lipid A analog, such as one of those described above, or a combination thereof) and an aPC. Standard methods for preparing and formulating drugs for administration by, for example, infusion are well known in the art and can be used in the invention. The antiendotoxin drug can be formulated, for example, by dissolving 35.4 mg of drug substance in 52.1 ml 0.01N NaOH, stirring for one hour at room temperature, and diluting into phosphate-buffered lactose. After adjusting the pH to 7.3 and diluting the drug to a final concentration of 0.1 mg/ml, the solution can be filter-sterilized and lyophilized. An example of a formulation of antiendotoxin drug product in 1 ml vials is shown below.

TABLE 1

| Material | amount |
|---|---|
| E5564 | 10 mg |
| NaH₂PO_{4.}4H₂O | qs |
| NaOH | qs |
| Lactose hydrous | 400 mg |
| Na₂HPO_{4.}H₂O | 1.8 mg |
| sterile water | 4 ml |

Activated Protein C can be formulated, for example, as a stable lyophilized formulation containing about 2.5 mg/ml aPC, 15 mg/ml sucrose, 20 mg/ml NaCl, and sodium citrate buffer. Additional appropriate formulations of these drugs, either alone or in combination, can readily be determined by those of skill in this art (see, e.g., Remington's Pharmaceutical Sciences (18^{th} edition), ed. A. Gennaro, 1990, Mack Publishing Company, Easton, Pa.).

Other embodiments are within the following claims.

What is claimed is:

1. Use of an antiendotoxin compound and activated Protein C in the prevention or treatment of septic shock or endotoxemia in a patient.

2. The use of claim 1, wherein said antiendotoxin compound is a Lipid A analog.

3. The use of claim 2, wherein said antiendotoxin compound is of the formula: 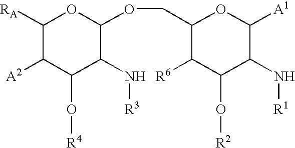
where R¹ is selected from the group consisting of 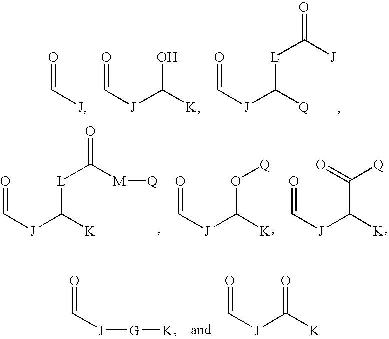
where each of J, K, and Q, independently, is straight or branched C1 to C15 alkyl;
L is O, NH, or CH₂; M is O or NH; and G is NH, O, S, SO, or SO₂;
R² is straight or branched C5 to C₁₋₅ alkyl;
R³ is selected from the group consisting of straight or branched C5 to C18 alkyl, 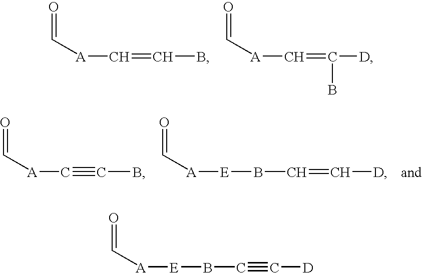
where E is NH, O, S, SO, or SO₂; each of A, B, and D, independently, is straight or branched C1 to C15 alkyl;
R⁴ is selected from the group consisting of straight or branched C4 to C20 alkyl, and 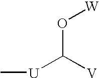
where each U and V, independently, is straight or branched C2 to C15 alkyl and W is hydrogen or straight or branched C1 to C5 alkyl;
R_{A} is R⁵ or R⁵—O—CH₂—, R⁵ being selected from the group consisting of hydrogen, J′, -J′-OH, -J′-O-K′, -J′-O-K′-OH, and -J′-O—PO(OH)₂, where each of J′ and K′, independently, is straight or branched C1 to C5 alkyl;
R⁶ is selected from the group consisting of hydroxy, halogen, C1 to C5 alkoxy, and C1 to C5 acyloxy;
A¹ and A², independently, are selected from the group consisting of OH, 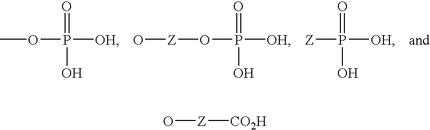
where Z is straight or branched C1 to C10 alkyl;
or a pharmaceutically acceptable salt thereof.

4. The use of claim 3, wherein said antiendotoxin compound has the following structure:

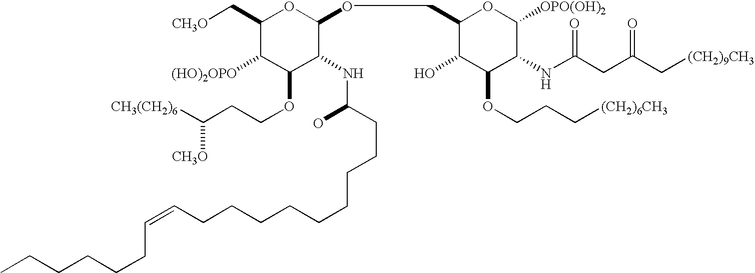

5. The use of claim 1, wherein said activated Protein C is recombinant human activated Protein C.

6. The use of claim 1, wherein said antiendotoxin compound and said activated Protein C are administered to said patient by continuous infusion, bolus, or intermittent infusion.

7. The use of claim 1, wherein said patient is a surgical patient.

8. The method of claim 7, wherein said surgical patient is a cardiac surgical patient.

9. The method of claim 1, wherein said patient has or is at risk of developing endotoxemia, sepsis, or septic shock.

10. A pharmaceutical composition comprising an antiendotoxin compound and an activated Protein C.

11. The composition of claim 10, wherein said antiendotoxin compound is of the formula: 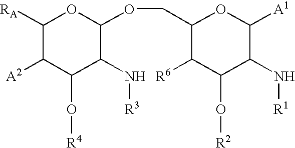
where R¹ is selected from the group consisting of 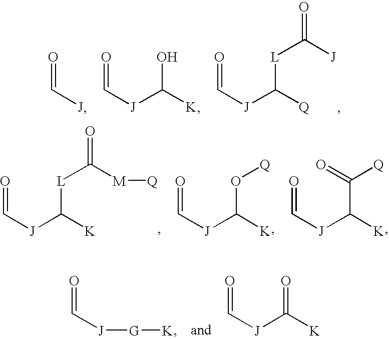
where each of J, K, and Q, independently, is straight or branched C1 to C15 alkyl;
L is O, NH, or CH₂; M is O or NH; and G is NH, O, S, SO, or SO₂;
R² is straight or branched C5 to C15 alkyl;
R³ is selected from the group consisting of straight or branched C5 to C18 alkyl, 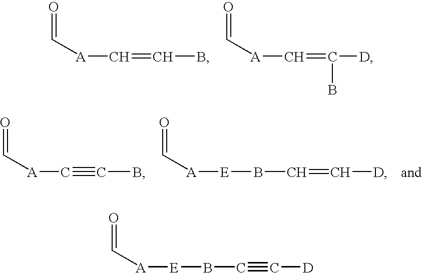
where E is NH, O, S, SO, or SO₂; each of A, B, and D, independently, is straight or branched C1 to C15 alkyl;
R⁴ is selected from the group consisting of straight or branched C4 to C20 alkyl, and 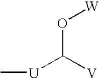
where each of U and V, independently, is straight or branched C2 to C15 alkyl and W is hydrogen or straight or branched C1 to C5 alkyl;
R_{A} is R¹ or R⁵—O—CH₂—, R⁵ being selected from the group consisting of hydrogen, J′, -J′-OH, -J′-O-K′, -J′-O-K′-OH, and -J′-O—PO(OH)₂, where each of J′ and K′, independently, is straight or branched C1 to C5 alkyl;
R⁶ is selected from the group consisting of hydroxy, halogen, C1 to C5 alkoxy, and C1 to C5 acyloxy;
A¹ and A², independently, are selected from the group consisting of 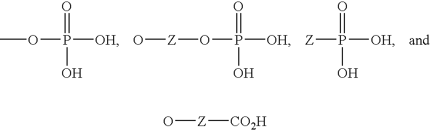
where Z is straight or branched C1 to C10 alkyl;
or a pharmaceutically acceptable salt thereof.

12. The composition of claim 11, wherein said antiendotoxin compound has the following structure:

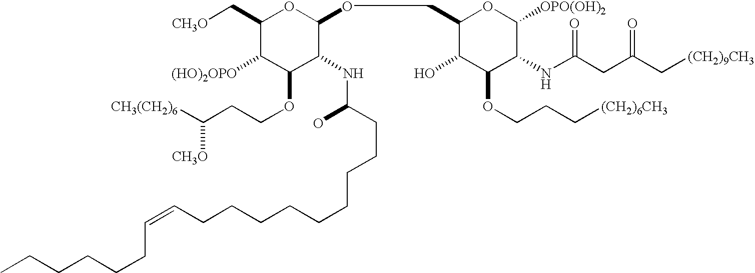

13. The composition of claim 11, wherein said activated Protein C is recombinant human activated Protein C.

14. A method of preventing or treating septic shock or endotoxemia in a patient, said method comprising administering an antiendotoxin compound and an activated Protein C to said patient.
